(19) United States
(12) Patent Application Publication
(10) Pub. No.: US 2018/0008381 A1
(43) Pub. Date: Jan. 11, 2018

(54) COATING OF DENTAL PROSTHETIC SURFACES COMPRISING A DISTINCT LAYER OF A SYNTHETIC HYDROXYAPATITE

(71) Applicant: KULZER GMBH, Hanau (DE)

(72) Inventors: Markus BALKENHOL, Neuberg (DE); Marcus HOFFMANN, Usingen (DE); Andrea LEYER, Bad Orb (DE); Susanne BUSCH, Wehrheim (DE)

(73) Assignee: KULZER GMBH, Hanau (DE)

(21) Appl. No.: 15/548,273

(22) PCT Filed: Feb. 3, 2016
(86) PCT No.: PCT/EP2016/052286

§ 371 Date: Aug. 2, 2017

(30) Foreign Application Priority Data
Feb. 4, 2015 (DE) 10 2015 101 626.5

(51) Int. Cl. A61C 8/00; A61C 8/02; A61F 2/30; A61C 13/24; A61C 13/09; A61C 13/271; A61L 27/32; A61F 2/28

(52) U.S. Cl. CPC A61C 8/0013; A61C 13/26; A61C 5/20; A61C 8/0006; A61C 8/0016; A61C 8/0022; A61L 27/32; A61F 2/30771; A61C 5/70; A61C 13/24; A61C 13/09; A61C 8/0074; A61F 2002/3069; A61F 2002/30838; A61F 2002/2817; A61L 2400/18; A61L 2430/02

(57) ABSTRACT

Subject matter of the invention are prosthetic mouldings, which have, at least area by area, at least one layer of biomimetic apatite selected from fluorapatite, hydroxylapatite or their mixtures on their surface, wherein the surface of the mouldings has micromechanical anchoring positions at least in this area to improve mechanical connection of apatite to the surface. Another subject matter of the invention are mouldings for use in dental, prosthetic treatment for tooth loss, in particular for cellular attachment of cells to prosthetic mouldings. Moreover, subject matter of the invention is the method for the production of the prosthetic mouldings.

Subject matter of the invention are prosthetic mouldings, which have, at least area by area, at least one layer of biomimetic apatite selected from fluorapatite, hydroxylapatite or their mixtures on their surface, wherein the surfaces of the mouldings have micromechanical anchoring positions at least in this area to improve mechanical connection of apatite to the surface. Another subject matter of the invention are mouldings for use in dental, prosthetic treatment for tooth loss, in particular for cellular attachment of cells to prosthetic mouldings, preferably an attachment of cells via hemidesmosomes. Moreover, subject matter of the invention is the method for the production of the prosthetic mouldings.

Frequently, a remaining gap between implant post and surrounding gums is a problem of fixed, permanent dental prostheses, such as implant-supported prosthetic restorations. Infections of the bone and the surrounding soft tissue are triggered by this gap over and over. Said problem is known under the term Periimplantitis or periimplant infection. In order to minimize the risk of infection, an immediate functional and structural linkage between the implant and the surrounded bone tissue (osteoblasts/bone cells) is aspired subsequent to an implantation. An implant, thus grown in, is osseointegrated. Therefore, adhesion and activity of osteoblasts on implant surfaces shall be increased to achieve accelerated implant integration. Osseointegration of an implant may be checked by means of ultrasound and radiography. In addition, an osseointegrated implant is immovable.

EP0125203 discloses a sintered, porous hydroxylapatite sleeve, which can be put on an implant post. A sleeve blank is produced from pressed amorphous material, probably a calcium phosphate (Ca₃(PO₄)₂), and is then sintered to transform the amorphous material into crystalline. It is emphasized that ingrowth of the bone tissue and the periosteum into the porous, crystalline hydroxylapatite layer ensues. The crystalline apatites produced according to the method of EP0125203 have considerably greater crystal size than biomimetically deposited apatite.

EP0657178A2 discloses a method for the production of implant ceramic material with hydroxylapatite. Here again, a method is disclosed in which the material to be used is sintered. Starting material is the spongiose material in the area of the joints of cattle. The inorganic material of the joints is sintered at high temperatures of 650 to 1250° C. The very high temperatures are required for accelerating crystal growth.

WO2011/022642A1 relates to nanoscale hydroxylapatite coatings (HAp) on orthopedic implants in a mixture with ZnO, which are applied by means of ESC depositing. The document refers to a multitude of attempts to produce HAp on metallic surfaces having acceptable properties of a lasting coating. A major disadvantage of amorphous calcium phosphate is its solubility in body fluids so that HAp is degraded over time. The method of WO2011/022642 discloses a method comprising milling steps with a ball mill and “supersonic jet milling” a jet mill. Deposition of nanoparticular particles is performed by spraying in an electrostatic field (electrospraying). Subsequently, the coating is sintered.

WO2011/049915A2 discloses electrochemical deposition of hydroxylapatite on implants from an aqueous solution. The method is limited to electroconductive material surfaces.

Object of the present inventions was the provision of prosthetic mouldings, which do not have the disadvantages of the state of the art. In addition, the object was to provide prosthetic mouldings which allow cellular attachment of cells of the gums or mucosal cells of the gums, respectively, via hemidesmosomes or other biological mechanisms to areas of the prosthesis contacting it. Therefore, the object of the invention was to provide prosthetic mouldings, which enable biological linkage or biological attachment, respectively, with the cells surrounding them. A further object was to provide crystalline, biomimetic apatite on prosthetic mouldings, in particular also on non-electroconductive surfaces, which can coalesce with surrounding cells, in particular with gums or epithelial cells, fibroblasts and/or osteoblasts.

The objects of the inventions are solved by a prosthetic moulding according to claim 1 as well as a method for the production of the prosthetic moulding according to claim 9 and the use of such a prosthetic moulding. Preferred embodiments of the inventions are disclosed in detail in the subclaims as well as in the specification. Prosthetic mouldings according to the invention also comprise surgical mouldings. Implants, implant abutments as well as all components connectable to the implant are more particularly preferred prosthetic mouldings.

Subject matter of the invention is a prosthetic moulding, wherein the moulding has, at least area by area, at least one layer of biomimetic apatite selected from fluorapatite, hydroxylapatite or their mixtures on its surface, wherein the surface has micromechanical anchoring positions at least in this area. Preferably, the surface additionally has chemical anchoring positions in this area, this surface is then considered to be activated. Chemical anchoring positions shall be understood to mean areas on the surface, which encourage biomimetic crystallization of apatite, such as, for example, areas of the surface having hydroxy groups.

The invention also relates to a moulding on which the biomimetic apatite is mechanically coalesced with the micromechanical anchoring positions, in particular in the form of a porous surface. It may also be preferred for the surfaces of the prosthetic mouldings, in particular in the area of the micromechanical anchoring positions, to be coated with TiN and/or ZrN or another physiologically compatible nitride. For example, this includes boron nitride, silicon nitride, aluminium nitride.

Micromechanical anchoring positions serve to improve mechanical connection of apatite to the surface. For example, biomimetic apatite may crystallise into a porous or on a roughened surface thus effecting mechanical anchoring of apatite on the surface.

Subject matter of the invention is a moulding for use in dental, prosthetic treatment for tooth loss, in particular for cellular attachment of cells synonymously cell adhesion, preferably for attachment of prosthetic mouldings via hemidesmosomes. The use for attachment of cells, such as epithelial cells or fibroblasts, to prosthetic mouldings is particularly preferred. For lasting, biological integration of implants in patients, cellular attachment of cells via hemidesmosomes or other biological mechanisms to prosthetic mouldings is aspired. Dental hemidesmosomal attachment of dental, prosthetic mouldings is important for regeneration of the gingiva/the gums on the basis of epithelial proliferation and its attachment to the prosthetic moulding, in order to form lasting biological connection to the implant and to minimize marginal gaps in this way. Hemidesmosomes are adhesion complexes which mediate adhesion of epithelial cells to an extracellular matrix. The adhesion complexes of hemidesmosomes feature a typical structure of intracellular plaque proteins, intermediary filaments and transmembrane contact proteins.

Subsequent to an implantation, it is aspired that the periimplant tissue may be biologically connected to the implant surface, e.g. the surface of the prosthetic moulding. Said connection does not succeed for common prosthetic mouldings due to rejection reactions of the body to exogenous materials. In contrast thereto, biomimetically deposited apatite provides an ideal basis for cellular attachment of cells, since biomimetic apatite quasi presents “own” tissue to epithelial cells or fibroblast of the gingiva and to the marginal, parodontous tissue. Biomimetic apatite comprising hydroxylapatite, fluorapatite and mixtures thereof is recognized by cells, in particular by gingiva epithelial cells and/or fibroblasts, as “natural tooth surface”. Whereas in the case of bone substitute materials based on apatite dissolution and remineralization of the bone substitute material is desired, it is important for an implant coating that the cells recognize the apatite surface as being endogenous and do not dissolve it, since otherwise the implant, here the titanium, would be uncovered again. Fluorapatite is sufficiently similar to human enamel to be recognized as being endogenous, but is not being dissolved.

According to the invention, the biomimetic apatite, comprising hydroxylapatite, fluorapatite and mixtures thereof is crystalline. Needle-shaped biomimetic apatite is obtained, wherein organic compounds of the gel forming agent, preferable of the gelatine, are embedded between the layers or in cavities. The content of the gel forming agent in the apatite may be from 0.001 to 10% by weight, in particular 0.5 to 5% by weight, based on the total composition of biomimetic apatite. Biomimetic apatite is very similar to the apatite of tooth enamel.

The mouldings according to the invention coated with biomimetically deposited apatite enable apposition of periimplant soft tissue on the basis of unimpaired cellular attachment via hemidesmosomes or other biological mechanisms of gingiva epithelial cells, fibroblasts or other cells so that the passage moulding gums may be sealed. According to the invention, a biological barrier is formed in this way by the cellular attachment, which significantly reduces risks of periimplant inflammation by bacteria.

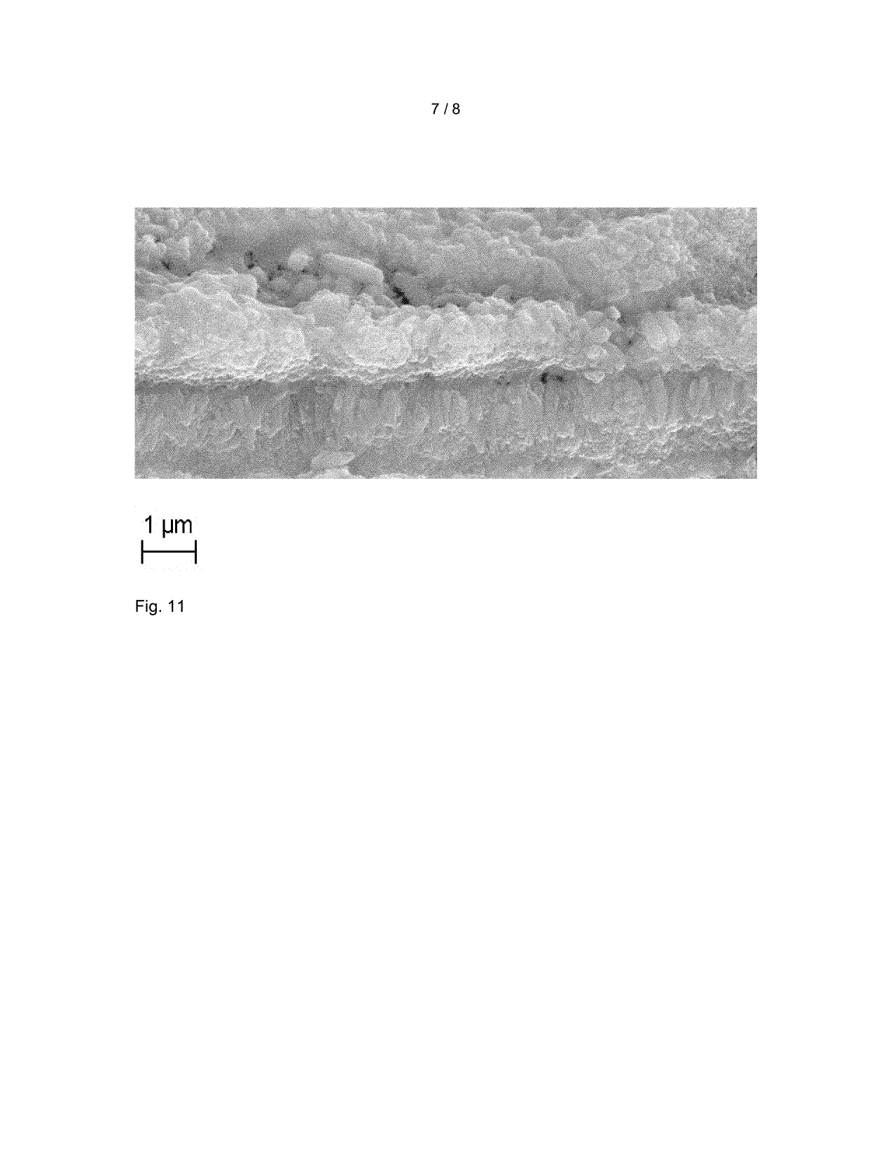

According to the invention, using the method according to the invention may create morphology and growth orientation of the apatite in accordance with natural tooth enamel. The biomimetic apatite preferably shows parallel orientation of the needle-shaped crystallites as it is observed near-surface in the case of tooth enamel. The crystallite size and orientation of biomimetically deposited apatite is exemplarily shown in FIG. 11. Basically, the crystallite size may vary, wherein larger crystallites have a diameter of about 100-250 nm and a length of about 500-1000 nm. In general, the crystallite size may deviate downwards as well as upwards, depending on crystallization conditions. According to an alternative of the invention, it may be preferred for the biomimetic apatite surfaces, which are grown onto the prosthetic moulding, to be polished, in order to reduce the surface roughness of the apatite surface (towards the epithelial cuff). The polished apatite surface preferably has low roughness of +/−0.005 to 10 μm, in particular of 0.05 to 5 μm, particularly preferably of 0.005 to 2 μm, whereas an unpolished apatite surface may have a roughness of greater than 10.5 μm. Thus, a sample having a difference of about 35 to 40 μm in height of the grown apatite layer has been measured. This is done in order to minimize bacterial adherence on the apatite surface.

According to a particularly preferred embodiment of the invention biomimetic apatite comprises a total content of C,H,N-atoms, which does not arise in electrolytically deposited apatite or sputtered apatite. Preferably, the total content of C,H,N-atoms is in the range of 0.01% by weight to 10% by weight, in particular of 0.05 to 5.0% by weight, particularly preferably about 2.3% by weight having a variance of plus/minus 0.5% by weight. The biomimetic apatite may also be deposited as biomineralised apatite from collagen and may then comprise components of collagen, denatured collagen, amino acid derivatives or proteins. The biomimetic apatite preferably comprises amino acids, amino acid derivatives, proteins, denatured collagen between the apatite layers and/or in cavities. The apatite, in particular between the apatite layers and/or in cavities, may also have components of collagen, denatured collagen, gelatine, protein chains and/or gelatine glycerin gel. Preferably, biomimetic apatite comprises from 0.001% by weight to 15% by weight collagen, denatured collagen, proteins and/or amino acid derivatives, in particular from 0.01% by weight to 10% by weight, preferably from 0.1 to 5% by weight, particularly preferably from 0.5 to 5% by weight, based on the total composition. According to a particularly preferred alternative, the moulding comprises biomimetic apatite or fluorapatite having a content of carbon and, optionally, a content of nitrogen. Preferably, the content of carbon is in the range of 0.25 to 2.5% by weight, preferably of 0.25 to 1.0% by weight and, optionally, the content of nitrogen in the range of 0.09 to 0.9% by weight. The content of denatured collagen or of gelatine, respectively, may be detected via determination of carbon and/or nitrogen content, for example by means of elementary analysis. It shall be assumed that embedding of the gel forming agent, in particular of the gelatine, ensues between the apatite layers or in cavities of the apatite. Thus, fragments having MM-peaks having a molecular weight of 18 and 44 (CO₂ and water), the degradation products of gelatine, could be detected by DTA-MS analysis, DSC-MS analysis. Heating of the layers to 1000° C. can result in blackening, since gelatine is burned to carbon. Detection of crystalline apatite or crystalline fluorapatite may be ensued by means of XRD.

The prosthetic moulding comprises, preferably in an area which later contacts epithelial cells, fibroblasts, other cells, the gums, the gingiva—such as, for example, gingiva epithelial cells or the area of the epithelial cuff (junctional epithelium), one apatite layer. Said apatite layer may comprise one or a multitude of apatite layers, such as 1, 2, 3 to 50 to an infinite number of layers. Depending on production method, one apatite layer or a multitude of apatite layers is applied on the area of the moulding. According to the invention, the prosthetic moulding is encompassed in the area of the apatite layer by the gingiva as epithelial cuff (junctional epithelium) so that the entry point of the implant or the prosthetic moulding, respectively, is sealed.

According to a particularly preferred embodiment of the invention the layer thickness of the biomimetic apatite comprising at least one or multiple apatite layers is from 100 nm to 1 mm, in particular form 500 nm to 800 μm, 1 μm to 500 μm are preferred, layer thicknesses of 10 μm to 500 μm are particularly preferred. Alternative layer thicknesses made of biomimetic apatite are in the range of 10 μm to 100 μm, of 20 μm to 200 μm, of 50 μm to 100 μm, of 100 μm to 200 μm, of 100 μm to 400 μm or of 1 μm to 50 μm. Medium thicknesses of biomimetic apatite in the range of 5 μm to 200 μm, preferably of 10 μm to 200 μm are further preferred.

The prosthetic mouldings according to the invention generally comprise all prosthetic mouldings, such as medical, orthopedic, orthodontic, dental mouldings as well as prosthetic moulding for anchoring, such as implants, screws, nails, surgical plates, as well as prosthetic mouldings as bone substitute, such as orthopedic prostheses, joint prostheses, such as joint endoprostheses, revision total joint endoprostheses, bone prostheses, vertebral bodies, spinous process or parts thereof. Preferred prosthetic mouldings comprise enossal implants, dental, enossal (intraosseous) implants, connecting element, mounting element, dental sleeve, abutment, superstructure/suprastructure, part of a dental prosthesis, total denture, orthopedic prosthesis or parts thereof, artificial tooth, veneer, inlay, onlay, dental supporting structure, bridge, crown, relining, denture saddle and/or spacer. An enossal implant is particularly preferred as a moulding; in particular dental, enossal tooth implant, an implant post; a connecting element for an implant, a mounting element, such as an abutment for a dental implant as well as surgical implants and all parts which may be assigned to an implant or a superstructure. The person skilled in the art knows that, according to the invention, all appropriate mouldings made of every physiologically compatible material may be provided with a biomimetic apatite layer as prosthetic moulding.

In general, prosthetic mouldings may be made from all appropriate physiologically compatible materials. The moulding may particularly preferably comprise a metallic material, an alloy, a dental alloy, ceramic, a hybrid material. Preferably, the moulding may be formed of titanium, titanium alloy, titanium oxide, cobalt-chromium allay, CoCrMo alloy, gold, dental ceramic, zirconium oxide, in particular ZrO₂, lithium disilicate, polymer, polymer mixture, dental prosthetic plastic.

Besides essentially biological components, such as proteins, collagen, denatured collagen, being biomimetically encompassed or being encompassed in the crystals or between the crystals by precipitation or crystallization of apatite, it is also preferred for the moulding to comprise amino acids as well. Therefore, the invention also relates to a moulding comprising a layer of biomimetic apatite which comprises amino acids, amino acid derivatives, proteins, denatured collagen and/or gelatine glycerin gel.

Moreover, the moulding may have, at least area by area, microretentions as micromechanical anchoring positions in the surface topography at its surface. Said microretentions serve as mechanical linkage positions and preferably comprise a porous, cracked and/or rough surface topography. Particularly preferably, the surface is porous at least in this area or to the whole surface. Said surface topography or the mechanical linkage positions, respectively, may be created by mechanically treating at least one area of the surface of the moulding, in particular by means of sandblasting, for example with corundum, hazelnuts or other common media. The surface may also be treated and/or activated chemically, such as by etching by means of acid or alkali, electrochemically, for example in an electrolytic process, and/or in a plasma process. The surface may also be treated and/or activated in a method which combines the afore-mentioned processes, wherein micromechanical anchoring positions are formed due to the treatment. Formation of micromechanical anchoring positions being additionally chemically activated is particularly preferred. This comprises, for example, formation of a porous and/or rough surface which is subsequently chemically etched forming chemical anchoring positions. Said chemical anchoring positions may encourage biomimetic crystallisation of apatite as well as its strong linkage to the moulding.

According to a further alternative, the moldings may also be produced in a generative or ablative method in a manner allowing the surface to have micromechanical anchoring positions. A possible generative method comprises laser sintering, an ablative method may comprise milling, optionally combined with an electrolytic process to obtain a surface having anchoring positions, such as, preferably, a porous surface. Typically, titanium dioxide or another metallic alloy may be electrochemically treated with the formation of gas or corrosion of the alloy. The surface comprising, at least area by area, micromechanical anchoring positions, preferably comprises a rough and/or porous surface topography. The surface comprising anchoring positions may therefore have a different chemical constitution than the remaining part of the prosthetic moulding. A titanium alloy having, area by area, a porous, oxidic surface may be an example. A surface should be understood as being porous if the surface has undercut and/or pores.

According to the theory of the invention, without being limited to it, the biomimetic apatite is deposited according to the method according to the invention at the anchoring positions of the surface, preferably in and/or at a porous surface so that the biomimetic apatite is firmly coalesced with the anchoring positions of the surface, in particular of the porous surface.

Moreover, subject matter of the invention is a prosthetic moulding or a part thereof having a) a surface having, at least area by area, micromechanical anchoring positions, in particular microretentions in the surface topography and/or chemical anchoring positions, in the area which is later arranged in the area of the gums, i.e. at the passaging point from mouth into jaw,

b) a surface having, at least area by area, micromechanical anchoring positions in the area, which is later arranged in the area of epithelial cells, fibroblasts, the gums, the gingiva, in particular the gingival epithelial cells, or the area of the epithelial cuff (junctional epithelium), wherein the prosthetic moulding is selected from connecting element, mounting element, abutment, implant, upper, outer prosthetic structure, as well as further mentioned prosthetic parts.

According to an alternative of the invention, the biomimetically deposited apatite layer on the prosthetic moulding may be superficially smoothed and/or polished. The person skilled in the art knows common methods for smoothing and/or polishing in dental field.

Another subject matter of the invention is a method for the deposition of biomimetic apatite on a prosthetic moulding, in particular a dental prosthetic moulding, selected from fluorapatite, hydroxylapatite or their mixtures, comprising the steps:

(i.a) providing a prosthetic moulding, wherein at least one area of the surface of the moulding has micromechanical anchoring positions, preferably the micromechanical anchoring positions are obtainable by mechanical, chemical, electrochemical treatment, plasma treatment, etching, depositing, or by combination of the mentioned treatments, or
(i.b) treating at least one area of the surface of a prosthetic moulding, wherein the treating preferably comprises mechanical, chemical, electrochemical treatment, plasma treatment, etching, depositing or combinations of the afore-mentioned treatments, optionally followed by cleaning, in particular washing of the surface, and obtaining of micromechanical anchoring positions, in particular micromechanical and, optionally, chemical anchoring positions, and optional
(ii) treating at least one area of the surface of a prosthetic moulding with a pretreatment composition having a defined pH value, in particular pH 8 to 10,
(iii) contacting at least this area, in particular the treated surface, of the prosthetic moulding comprising micromechanical anchoring positions with a compositions containing phosphate ions, which comprises a gel forming agent, preferably gelatine, whereby a gel layer is formed, the surface is preferably covered at least in part with the gel, wherein the covered areas of the surface are essentially totally and consistently covered with a gel layer, in particular with a gel layer of 0.001 mm to 10 mm, preferably of about 1 mm to 5 mm,
(iv) optionally applying a further layer forming a further gel layer, in particular by contacting the first gel layer with a further composition which comprises a gel forming agent, which is in particular free of phosphate ions, calcium and/or fluoride ions, preferably the gel layer has a thickness of 0.001 mm to 10 mm, preferably of about 1 mm to 5 mm, and
(v) contacting the first gel layer or the further gel layer with a composition containing calcium ions, whereby a gel layer is formed,
(vi) depositing biomimetic apatite selected form fluorapatite, hydroxylapatite or their mixtures, in particular crystalline biomimetic apatite, on the surface of the prosthetic moulding. Moreover, subject matter of the invention is a prosthetic moulding obtainable by the method according to the invention. Application time for depositing the apatite may last from 1 to 48 hours, single to multiple application each of 1 to 10 hours is preferred. In this context, it is particularly preferred for the method to be performed at a temperature of 20 to 40° C., preferably from 30 to 39° C.

Contacting with the gel layer may be ensued, for example, by wrapping gel threads or gel strips around an implant, and a multitude of gel threads form, side by side, a gel layer. For example, the threads may be wrapped around the prosthetic mouldings analogously to a thread spool. Likewise, spraying on, applying with a brush, applying from a nozzle or imprinting of the gel layer is possible.

Preferably, deposition of biomimetic apatite ensues with a layer thickness of at least 1 μm, preferably from 1 μm to 500 μm, preferably 2 μm to 200 μm, particularly preferably from 5 μm to 100 μm.

Treatment of the surface of a prosthetic moulding particularly preferably comprises an additional treatment of the surface having micromechanical anchoring positions with a pretreatment composition. Said composition may be applied on the surface and may affect it. The composition containing phosphate ions may be applied on the pretreatment composition. Alternatively, the pretreatment composition may be removed after an application time.

Availability of phosphate may be increased in the composition containing phosphate ions (first gel) preferably by addition of amino acids having additional basic groups. Furthermore, all substances having binding sites for calcium ions and phosphate ions without precipitating them or having toxic effects for the human organism are suitable to increase the solubility. This includes, for example, vitamins (e.g. ascorbic acid), oligopeptides, carboxylic acids, in particular, fruit acids, such as malic acid, citric acid, lactic acid or pyruvic acid or chelating agents, such as EDTA. According to the invention, it is proposed to combine 1-3 different gel layers, wherein their order is determined.

A two-gel-layers-method is proposed, in which the cover gel has inducing effect on mineralisation. Using thin, pre-fabricated gel films is more economical in an industrial process. Said gel films, for example, may be rolled onto the prosthetic mouldings. Alternatively, the prosthetic moulding may be guided over the composition, wherein the respective composition adhere to a defined area of the surface of the moulding or to a composition already applied. The moulding is preferably contacted with a heated composition, i.e. liquefied composition. The composition may then be solidified on the moulding by cooling. Alternatively, the moulding may be sprayed with a heated liquid gel solution. Wrapping the moulding with a pre-fabricated gel thread or gel strip is preferred as well.

The layer thickness of the phosphate gel layer or calcium phosphate gel layer preferably is from 50 μm to 1 cm, in particular from 100 μm to 1000 μm, preferably 150 μm to 500 μm. The concentration of the phosphate ion composition is from 0.01 mol/l to 2 mol/l, preferably 0.08 mol/l to 0.3 mol/l. The concentration of fluoride ions is from 0 mol/l to 0.3 mol/l, preferably 0.0001 mol/l to 0.05 mol/l. The concentration of calcium ions preferably is from 0.0001 mol/l to 0.1 mol/l in the composition. The layer thickness of the calcium gel preferably is from 50 μm to 5 mm, preferably from 300 μm to 2500 μm, preferably from 300 μm to 1500 μm.

It has surprisingly been found that a calcium-free, alkaline pretreatment composition containing fluoride ions has growth accelerating effect on formation of the apatite layer. Gelatine may be added to the composition in order to give a gel-consistency and to improve the adherence at the surface.

Treatment of at least one area of the surface of a prosthetic moulding may comprise mechanical, chemical, electrochemical treatment and/or treatment by means of a plasma process, preferably to create the micromechanical anchoring positions. Said treatment may be followed by another treatment with an acidic or preferably alkaline pretreatment composition to chemically activate and/or to clean the created micromechanical anchoring positions.

According to the invention, micromechanical anchoring positions are required to enable, on the one hand, preferably uniform distribution of the pretreatment composition on the surface of the moulding and/or, on the other hand, firm and lasting mechanical anchoring of biomimetic apatite to the moulding by crystallising the apatite into the porous surface of the moulding. The surface of the moulding has micromechanical anchoring positions, preferably being in the form of a porous surface. In addition, canal-like structures may be formed as anchoring positions in the surface. Pore diameters of the porous surface of the prosthetic moulding, in particular of implants in the infracrestal area, preferably are in the range of about 0.5 to 150 μm, particularly preferably the pore diameter according to an alternative is at 50 to 120 μm, preferably at 65 to 105 μm. According to a further alternative, in order to deposit biomimetic apatite it is preferred for the pore diameter of the prosthetic moulding to be a multiple of the crystallite diameters. This facilitates crystallisation of biomimetic apatite into the porous surface thus causing firm mechanic anchoring. Therefore, it may also suffice and be preferred for pore diameters to be slightly smaller in the range of 0.2 μm to 70 μm, preferably 0.2 μm to 50 μm, particularly preferably of 0.2 to 20 μm, in particular in the range of 0.5 μm to 10 μm or of 0.5 to 5 μm. Further preferred is range of 1 μm to 10 μm. Average roughness of the surface of the area having micromechanical anchoring positions may be in the range of Rₐ (average roughness, line profile 2D) of Rₐ greater than or equal to 0.75 to 4 μm, in particular of 1.0 to 4.0 μm, of 1.0 to 3.0 μm. Mouldings produced by means of CNC milling machines have a Rₐ value of about 0.29 μm. Mean roughness Sₐ (surface profile, 3D) of the surface having micromechanical anchoring positions preferably is Sₐ greater than or equal to 1.2 μm to 4.0 μm, in particular greater than or equal to 1.25 μm to 3.0 μm in surfaces having micromechanical anchoring positions. The mean surface roughness of a surface produced by means of a CNC milling machine is at less than 0.75 μm.

The pretreatment composition according to the invention preferably activates the surface comprising micromechanical anchoring positions or is able to form the micromechanical anchoring positions by an etching, depending on the material of the surface, thus improving biomimetic deposition of apatite and/or fluorapatite. Said activation may comprise formation of oxidic areas, formation of hydroxy groups and/or cleaning of the surface.

The alkaline pretreatment composition may comprise an alkaline pretreatment composition, in particular having a pH value of 8 to 14, in particular 9 to 14, preferably a pH value about 14. A sodium fluoride solution or a sodium fluoride gel, such as comprising 0.01 mol to 3.0 mol NaF is particularly preferred. Preferably, the pretreatment composition comprises about 0.1 mol to 2.0 mol NaF, a 0.5 molar sodium fluoride solution set to pH 14 and optionally having a content of gelatine is particularly preferred, preferably the content of gelatine is from 1 to 20% by weight, in particular 5 to 10% by weight, about 7.5% by weight gelatine are further preferred. The alkaline pretreatment composition remains on the surface of the prosthetic moulding. Subsequently, at first the gel containing phosphate ions and hereinafter the gel containing calcium ions is applied on the pretreatment composition.

According to the invention, the pretreatment composition is additionally used in the method of the invention, in particular for activating the surface having micromechanical anchoring positions or for forming the micromechanical anchoring positions, wherein the pretreatment composition has a pH value of 8 to 14, in particular pH 8 to 10 or pH 12 to 14, and preferably comprises fluoride ions, such as NaF, NH₄F or other soluble amino fluorides. Alkali hydroxides, such as NaOH, KOH or earth alkali hydroxides, such as Ca(OH)₂ etc. may be used for setting the pH value.

Due to use of the alkaline pretreatment composition, extremely thin films are reproducibly formed on the surface of the prosthetic moulding which particularly encourage the initial mineralisation. Depending on the ion content of the compositions containing phosphate ions and/or calcium ions, i.e. of the first or second gel, the pretreatment composition may comprise from 0.5 mol/l to 3 mol/l calcium ions, or from 0.0 mol/l to 1 mol/l phosphate ions, in particular 0.001 mol/l and/or from 0 mol/l to 1 mol/l fluoride ions. The pH value may be from 8 to 14, in particular pH 8 to 10.

An alkaline pretreatment composition with a 0.5 molar, aqueous sodium fluoride composition set to pH 14 and optionally comprising about from 0.1 to 7.5% gelatine is also preferred. An alkaline pretreatment composition with a 0.5 to 3.0 molar, preferably 1.0 to 3.0 molar, aqueous composition containing calcium ions set to pH 14 may also be used. Said composition is applied, area by area, to the surface of the prosthetic mouldings and, following this, humidity is blown away.

According to a preferred alternative of the method, in the method according to the invention (a) a composition containing phosphate ions is used, which comprises,

(a.1) at least one gel forming agent,
(a.2) water-soluble phosphates or phosphates being hydrolysable to water-soluble phosphate ions, in particular phosphate ions or hydrogen phosphate ions,
(a.3) optionally fluoride, (a.3.1.) optionally one or multiple amino acids,
(a.4) optionally a carboxylic acid or a puffer system of pH 4 to 7,
(a.5) optionally glycerin. This composition may also be referred to as first gel.

Moreover, according to a preferred alternative of the method, in the method according to the invention (b) a composition containing calcium ions is used, which comprised

(b.1) at least one gel forming agent,
(b.2) calcium ions
(b.3) optionally glycerin. This composition may also be referred to as second gel.

The compositions preferably have a pH value of 2 to 9, preferably of 3 to 8, particularly preferably of 4 to 7, further preferably of 4 to 6.

The compositions according to the invention may comprise, each independently, amino acids, derivatives of amino acids, proteins or denatured collagen.

Gel forming agents may be selected from denatured collagen, gelatine, gelatine glycerin gel, hydrocolloids, polypeptides, protein hydrolysates, polysaccharides, polyacrylates or mixtures comprising at least two of the mentioned gel forming agents. Preferably, the compositions comprise, each independently, gelatine and a polyol, preferably glycerin, their adducts and/or their reaction products.

The compositions according to the invention, in particular the composition containing phosphate ions and/or calcium ions, the pretreatment composition, preferably comprise, each independently, a content of water. The content of water may vary in the composition containing phosphate ions, in particular in the gel, from about 45 to 55% by weight and in the composition containing calcium ions, in particular in the gel, from about 30 to 40% by weight, based on the total composition.

Treating the surface of the moulding comprises mechanical, chemical, electrochemical treatment and/or treatment in a plasma process or a combination of the methods, forming at least one area of the surface of the prosthetic moulding having micromechanical anchoring positions. Surface treatment results in an increase of the surface, in particular a porous surface is formed. The surface thus treated is microretentive so that the biomimetic apatite may lastingly coalesce with the surface structure. Moreover, in the method according to the invention the surface of the moulding may additionally be activated mechanically, chemically and/or in a plasma process or by combination of the processes.

Another subject matter of the invention is the use of biomimetic apatite for coating, at least area by area, of prosthetic mouldings. According to the invention, biomimetic apatite shall be understood to mean apatite being deposited from gelatine or collagen, which comprises components of gelatine, collagen, denatured collagen, amino acid derivatives and proteins. Preferably, biomimetic apatite comprises from 0.001% by weight to 15% by weight collage, denatured collagen protein and/or amino acid derivatives, in particular from 0.01% by weight to 10% by weight, preferably from 0.1 to 5% by weight, particularly preferably from 1 to 10% by weight in relation to the total composition. Detection of the deposition of biomimetic apatite may be ensued on the basis of detection of the carbon content and/or the nitrogen content, not existing in sintered apatite. Furthermore, biomimetic apatite or fluorapatite forms macrocrystalline planar crystal structure, in whose pores and cavities gelatine is embedded, since the crystalline phase has been deposited from humid gelatine. In contrast thereto, apatites produced from apatite powder and subsequent sintering do not have biomimetic macrostructures. Sintered apatite is not being recognized by the body for apposition of periimplant soft tissue on the basis of unimpaired cellular attachment via hemidesmosomes or other biological mechanisms of gingiva epithelial cells, fibroblast or other cells. In addition, the afore-mentioned high sintering temperatures of about 1250° C. cause warping of the geometry of the implant basic structure in implants so that fit accuracy of the connection geometry of the sintered implant is not correct anymore.

Moreover, subject matter of the invention is the use of compositions containing phosphate ions and calcium ions, such as of a first gel and of a second gel, or of formulations containing these compositions, for biomimetic deposition of apatite on the surface of a prosthetic moulding, wherein the surface comprises micromechanical anchoring positions and/or wherein the surface of the moulding has been activated mechanically, chemically, electrochemically and/or by means of a plasma process prior to deposition. The activated surface comprises chemical anchoring positions, such as functional groups, which may be, for example, oxidic or hydroxy functional.

Furthermore, subject matter of the invention is the use of the afore-mentioned compositions for the deposition, at least area by area, of biomimetic apatite and/or essentially homogenous depositions, at least area by area, of apatite, in particular in this area. A deposition of apatite is considered to be essentially homogenous if the interested area of the surface of the prosthetic moulding is covered by at least 80% with an apatite layer, 90% are preferred, 90 to 100% are particularly preferred.

In a particularly preferred embodiment according to the invention the composition containing phosphate ions comprises: (i) water-soluble phosphates or phosphates being hydrolysable to water-soluble phosphate ions, in particular Na₂HPO₄, preferably the phosphate content in the composition is at 1 to 10% by weight, preferably from 2 to 8% by weight, particularly preferably from 5 to 8% by weight, (ii) a content of water or a mixture of water and an organic solvent, (iii) optionally at least one carboxylic acid, in particular a hydroxy carboxylic acid, such as lactid acid, and/or a buffer system, in particular a buffer system for setting the pH value is in the range of 2 to 8, in particular of 3.5 to 8, preferably of 3.5 to 6, particularly preferably about 4.5 plus/minus 1.0, in particular plus/minus 0.5. The content is based on the (HPO₄)²⁻ concentration being weighed in.

In a particularly preferred embodiment according to the invention the composition containing calcium ions comprises: (i) calcium ions or compounds releasing calcium ions, in particular calcium dichloride or hydrates thereof, preferably, additionally, calcium sulfate, nanoapatite, sodium carbonate or calcium oxalate, preferably the calcium content in the composition is at 1 to 10% by weight, preferably greater than or equal to 1.5 to 7.5% by weight, (ii) optionally water or a mixture of water and an organic solvent, and (iii) optionally at least one carboxylic acid, such as a hydroxy carboxylic acid, for example, lactic acid, and/or a buffer system. Fruit acids and alkali salts are preferably used for the production of the buffers. The content is based on Calcium (Ca²⁺). The pH value is preferably set to 5.5 with plus/minus 0.5.

It is further preferred for the compositions to comprise at least one water-soluble fluoride (F⁻), with fluoride ions, or one compound releasing fluoride. Particularly preferably, the composition containing phosphate ions has as further component (iv) at least one water-soluble fluoride or one compound releasing fluoride.

According to a preferred embodiment of the invention, the at least one water-soluble fluoride or the at least one compound releasing fluorides comprises (iv) at least one quaternary mono- or poly-ammonium compound having unsubstituted or substituted alkyl group, preferably having four substituted alkyl groups, wherein the at least one substituted alkyl group comprises hydroxy alkyl-, carboxy alkyl-, amino alkyl-groups having 1 to 25 C-atoms or organo functional groups interrupted by hetero atoms, having up to 50 C-atoms. Preferred ammonium compounds may comprise 1 to 20 quaternary ammonium functionalities, preferably 1, 2, 3, 4, 5, 6, 7, 8 ammonium functionalities, preferably Olaflur (N,N,N′-Tris(2-hydroxyethyl)-N′-octadecyl-1,3-diaminopropanedihydrofluoride) is used as water-soluble fluoride. Amino fluorides, such as Olaflur, Decaflur, ethanol amino hydrofluoride, an organo functional amino compound releasing fluorides, or an antiseptic agent on the basis of organo functional amino compounds releasing fluorides, such as, in particular, fluorides of N-Octyl-1-[10-(4-octyliminopyridine-1-yl)decyl]pyridine-4-imine, cetylpyridinium fluoride, or water soluble inorganic fluorides, such as alkali fluoride, sodium fluoride, potassium fluoride, tin fluoride, ammonium fluoride, or inorganic fluorides releasing fluorides, such as zinc fluoride, zinc hydroxy fluoride are preferred as well.

as gel forming agent according to the invention At least one gel forming agent selected from gelatine, denatured collagen, hydrocolloids, polypeptides, protein hydrolysates, synthetic polyamino acids, polysaccharides or mixtures comprising at least two of the mentioned gel forming agents may preferably be present in the respective composition. Gelatine in which a plasticizer such as glycerin or another polyol is added according to the invention is preferably used. Addition of the plasticizer improves handling properties of the gelatine. Compositions according to the invention preferably comprise gelatine and a polyol, such as glycerin and/or their reaction products as gel forming agents, optionally in the presence of water. Alternatively, gelatine and a plasticizer, such as sorbitol, may also be used. The plasticizer ensures increase of the melting range by forming intermolecular hydrogen bonds.

The gel forming agent according to the invention is gelatine, preferably (denatured collagen, animal protein, protein), particularly preferably a collagen being acidly hydrolysed, or gelatine and a polyol, such as glycerin, is used. Alternatively, casein, starch, cellulose, HPMC, gums arabic, galactomannan, guar gums, konjac, xanthan gums, calcium alginate, dextran, scleroglucan, pectin, carrageenan (K-, I- and λ-carrageenan), agar agar, alginate, alginic acid, sodium alginate, calcium alginate, tragacanth may be used as gel forming agent, wherein gelatine or mixture with gelatine are preferred.

Carboxylic acids are preferably selected form fruit acids, such as α-hydroxy carboxylic acids, such as malic acid, citric acid, glycol acid, lactic acid and tartaric acid; amino acids, fatty acids, hydroxy carboxylic acids, dicarboxylic acids and mixtures comprising at least two of the mentioned acids, and/or the buffer system comprises carboxylates of alkyl carboxylic acids, fatty acids, fruit acids, fumarates, amino acids, hydroxy carboxylic acids, dicarboxylic acids and mixtures comprising at least two of the mentioned acids, or phosphate buffer. Alkali salts and/or earth alkali salts or zinc salts are advantageously used for the buffer systems.

The buffer systems comprise EDTA, TRIS: Tris(hydroxymethyl)aminomethane for pH 7.2 to 9.0, HEPES: 4-(2-Hydroxyethyl)-1-piperazineethane sulfonic acid for pH 6.8 to 8.2, HEPPS: 4-(2-Hydroxyethyl)-piperazine-1-propane sulfonic acid for pH 7.3 to 8.7, barbital acetate buffer, MES: 2-(N-Morpholino)ethane sulfonic acid for pH 5.2 to 6.7, carbonic acid bicarbonate system for pH 6.2 to 8.6; neutral, carbonic acid silicate buffer for pH 5.0 to 6.2; weakly acidic, acetic acid acetate buffer for pH 3.7 to 5.7, phosphate buffer: NaH₂PO₄+Na₂HPO₄ for pH 5.4 to 8.0, ammonia buffer NH₃+H₂O+NH₄Cl for pH 8.2 to 10.2, citric acid buffer or citrate buffer. Particularly preferred buffer systems comprise lactic acid buffer systems, EDTA, or barbital acetate buffer and TRIS (Tris(hydroxymethyl)aminomethane) buffer.

Phosphates usable according to the invention for the production of the phosphate containing mineralisation matrices comprise phosphates, hydrogen phosphates or phosphates hydrolysable to water-soluble phosphate ions comprising a) alkali phosphates, earth alkali phosphates, dihydrogen phosphates, sodium dihydrogen phosphate, NaH₂PO₄, potassium dihydrogen phosphate, KH₂PO₄, hydrogen phosphates, dipotassium hydrogen phosphate, K₂HPO, disodium hydrogen phosphate, Na₂HPO₄, phosphate esters, monoesters, diesters and triesters of phosphates, sodium phosphate, Na₃PO₄, potassium phosphate, K₃PO₄, calcium dihydrogen phosphate, Ca(H₂PO₄)₂, monoesters, diesters and triesters of calcium hydrogen phosphate, CaHPO₄, calcium phosphate, Ca₃(PO₄)₂, and/or

b) the calcium ions or compounds releasing calcium ions comprise calcium chloride, calcium dichloride dihydrate, calcium salt of a carboxylic acid comprising alkyl carboxylic acids, hydroxy carboxylic acids, dicarboxylic acids, fruit acids, amino acids, such as calcium lactate, calcium gluconate, calcium lacto gluconate, calcium alginate, calcium L-ascorbate, compounds retardedly releasing calcium ions hardly soluble in water comprising calcium sulfate, calcium apatite, calcium carbonate, calcium oxalate, calcium phosphate, calcium alginate, preferably having a particle size of less than 100 μm, preferably about 10 μm, particularly preferably less than or equal to 5 μm, for example up to 1 μm or 50 nm, or preferably mixtures of calcium ions being water-soluble and hardly soluble in water or compounds releasing calcium ions. Compounds retardedly releasing calcium ions hardly soluble in water are added for texture improvement of partially sticky gels of compositions comprising highly water-soluble ions. In relation to the total composition of the composition, 1 to 50% by weight of calcium releasing compounds being hardly soluble in water may be used, preferably 5 to 30% by weight are used.

The composition containing phosphate ions comprises, among others, a water-soluble phosphate salt. For example, alkali salts, such as sodium phosphate or potassium phosphate, hydrogen phosphate or dihydrogen phosphate are suitable. The list is inclusive but non-exclusive. The concentration of phosphate salt in the composition, in particular gel, is from 0.05 mol/l to 4 mol/l composition, preferably 0.5 mol/l to 1.5 mol/l, particularly preferably about 1 mol/l plus/minus 0.5 mol/l. The composition containing phosphate ions further comprises a water-soluble fluoride salt, e.g. an alkali salt, or tin fluoride or Olaflur. The list is inclusive but non-exclusive. The concentration of the fluoride in the composition is from 0 to 6000 ppm by weight, preferably 200 to 4000 ppm by weight, particularly preferably 2500 to 4000 ppm by weight, or about 3000 ppm by weight plus/minus 500 ppm by weight. The pH value of the phosphate composition is preferably from 2.0 to 8.0, preferably from 3.5 to 5.5 and is set by an appropriate buffer system. Carboxylic acids, such as ascorbic acid, pyruvic acid, tartaric acid, acetic acid, lactic acid or malic acid, but also all other buffer system are particularly well suited. The concentration of the buffer is from 0.25 mol/l to 4.0 mol/l, preferably from 0.5 mol/l to 1.5 mol/l.

For the production of the compositions containing gel forming agent, gelatine or glycerin may be added to the respective composition. The amount of gelatine preferably is 25 to 40% by weight and the amount of glycerin 5 to 20% by weight, based on the total composition in a composition, in particular a water-containing composition. In order to mix the components homogenously, the composition is heated to 40 to 90° C., preferably to 50 to 70° C. The layer thickness of the phosphate ion composition in the method or of the composition on the intermediate is here from 50 μm to 3000 μm, preferably from 200 μm to 2000 μm, particularly preferably 300 μm to 1500 μm.

In preferred alternatives, the compositions comprise, each independently, 5 to 50% by weight gelatine, based on the total composition and 0 to 30% by weight glycerin, based on the total composition, 25 to 40% by weight gelatine and 5 to 20% by weight glycerin in the composition containing phosphate ions and 20 to 40% by weight gelatine and 15 to 25% by weight glycerin in the composition containing calcium ions are preferred. The gelatine containing compositions are preferably heated to 40 to 90° C. in order to mix the components homogenously, the temperature range of 50 to 70° C. is preferred. Subsequently, the compositions are allowed to cool down, wherein they are solidifying.

The composition containing calcium ions preferably comprises a water-soluble calcium salt, e.g. calcium chloride or calcium lactate or calcium gluconate or calcium lacto gluconate. The list is inclusive but non-exclusive. The concentration is from 0.1 mol/l to 2.0 mol/l, preferably from 0.5 mol/l to 1.5 mol/l. The pH value is from 4.0 to 14.0, preferably from 6.0 to 11.0 and is set by an appropriate buffer system. Carboxylic acids, such as ascorbic acid, pyruvic acid, tartaric acid, acetic acid, lactic acid or malic acid are particularly well suitable, but also all other buffer systems having suitable pKs value may be used.

The concentration of the buffer is from 0.1 mol/l to 3.0 mol/l, preferably from 0.25 mol/l to 1.0 mol/l. In this composition the content of gelatine preferably is 20 to 40% by weight in relation to the total composition and the amount of glycerin 15 to 25% by weight. Since the calcium gelatine composition is extremely sticky as well after gelling and thereby inconvenient in handling, a hardly soluble calcium salt is added for texture improvement. Calcium sulfate, calcium apatite, calcium carbonate, calcium oxalate are particularly well suited. The list is inclusive but non-exclusive.

The layer thickness of the composition containing phosphate ions in the method according to the invention or of the intermediate preferably is from 10 μm to 1 cm, in particular 10 μm to 5 mm or up to 3000 μm, preferably 100 μm to 3000 μm, particularly preferably 500 μm to 3000 μm, preferably 500 μm to 1500 μm.

For the production of the intermediates or for the procedure of the method, compositions not yet solidified, in particularly the first, second and optionally further gels, are moulded and subsequently solidified.

The compositions containing phosphate ions, calcium ions and fluoride ions according to the invention may be produced as disclosed in EP1509189A1, EP1927338A1, EP1927334A1 and/or EP1809233A1.

Therefore, another subject matter of the invention is a method, in which in at least one step a non-solidified composition is respectively applied on an area of the prosthetic moulding, optionally further non-solidified compositions may be applied on this composition.

Another subject matter of the invention is an intermediate, comprising a prosthetic moulding, wherein the moulding has, at least area by area, at least one gel layer (first gel layer) of a composition containing phosphate ions on its surface. Preferably, the intermediate optionally has a further gel layer on this first gel layer, and, optionally, a gel layer of a composition containing calcium ions. Thus, the intermediate according to the invention may have a first gel layer containing phosphate ions, a second gel layer and a third gel layer containing calcium ions. Alternatively, it has a first gel layer containing phosphate ions and a second gel layer containing calcium ions, or, alternatively, a gel layer containing phosphate ions and calcium ions. As described above, fluoride may be contained in at least one of the compositions.

The invention is elucidated in more detail with the figures, without limiting the invention to the subject matter of the figures.

The figures show schematically:

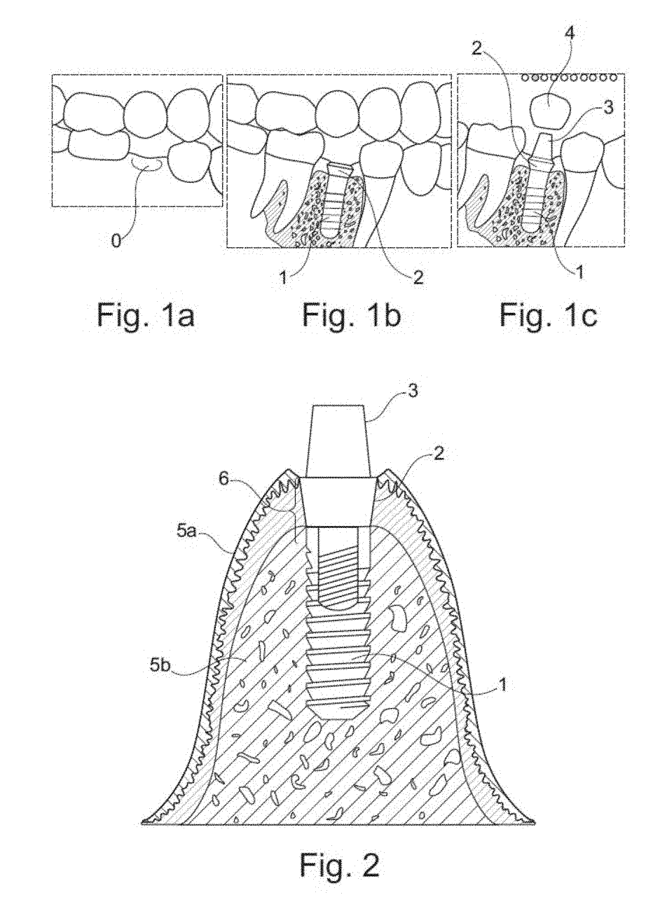

FIG. 1a: an edentulous area 0 in which a crown 4 shall be inserted,

FIG. 1b: the edentulous area in which an implant 1 with a connecting element 2 has been inserted in the bone,

FIG. 1c: implant 1 with connecting element 2, mounting element 3 and crown 4.

FIG. 2: cross section (without perspective) of a jaw area comprising gums 5a, gingiva 5a and jawbone 5b, showing the area 6 of connecting element 2 or implant 1 (prosthetic moulding, respectively) in the gums 5a of the epithelial cuff (junctional epithelium).

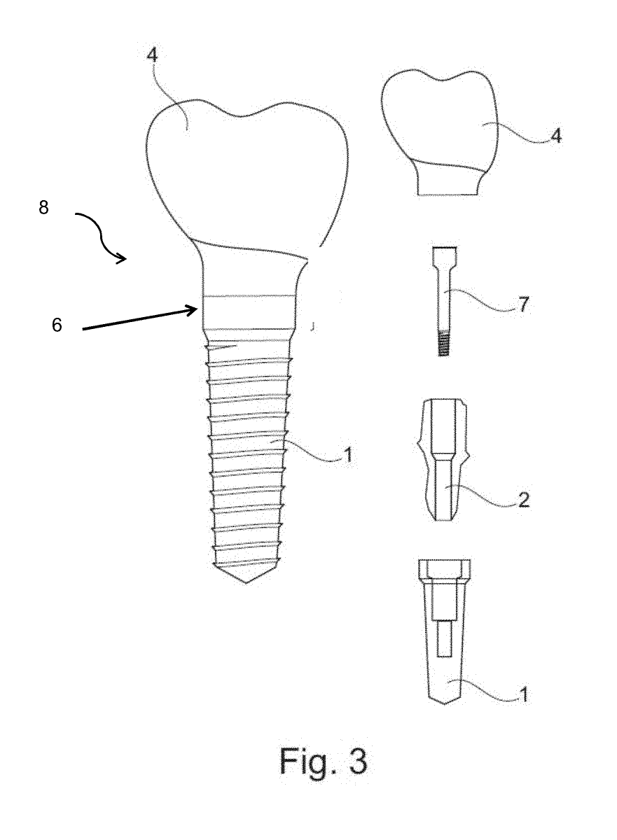

FIG. 3: Typical structure of a prosthetic tooth restoration 8 comprising an implant 1, a connecting element 2, the upper, outer prosthetic structure 4, e.g. dental crown, suprastructure optionally with outer coating, the area 6, in particular (junctional epithelium) of the implant and/or connecting element in the gums as well as a fixation screw 7.

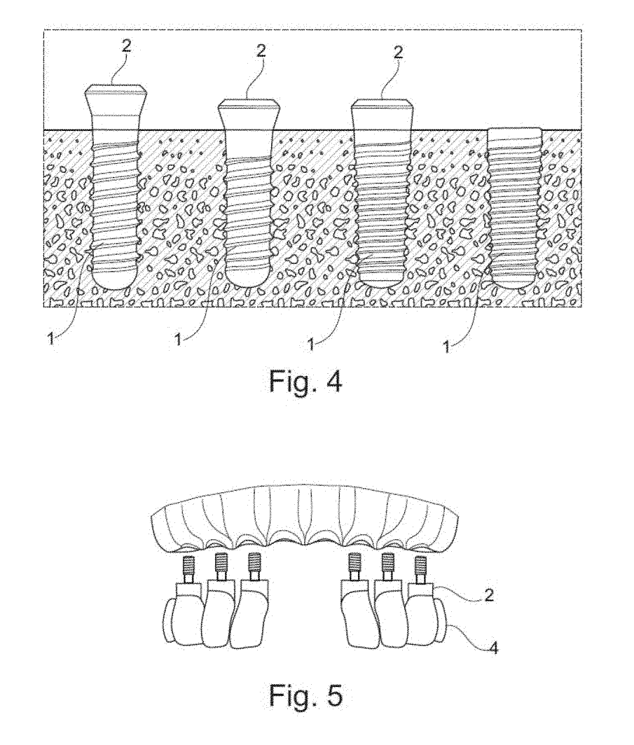

FIG. 4: Shows different installation situations of implants 1 in the jawbone with or without connecting element 2.

FIG. 5: Shows a multitude of prosthetic restorations with crowns 4 comprising prosthetic mouldings according to the invention, such as connecting element 2 (abutment, spacer, pillar, post, implant shoulder etc.) optionally with connecting screw.

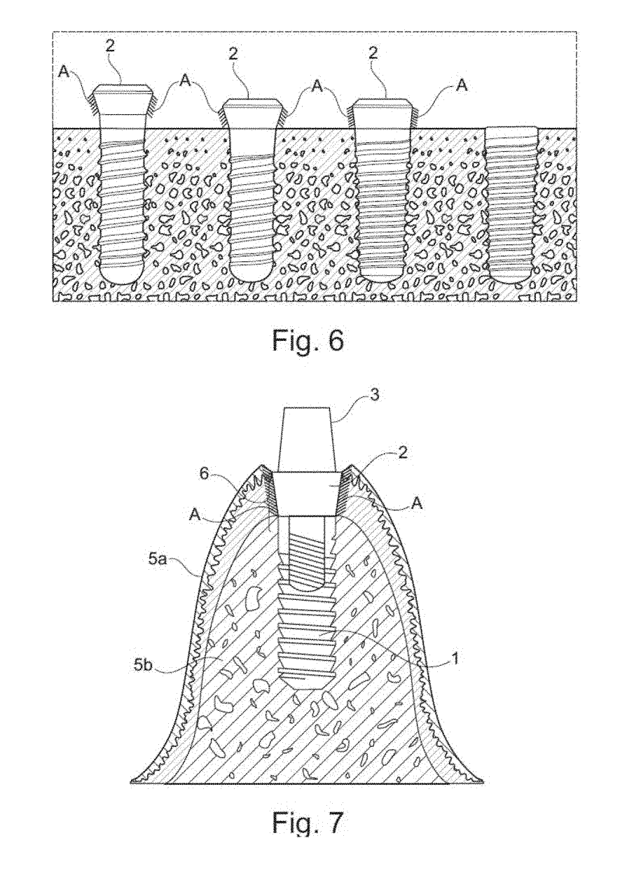

FIG. 6: Shows different installation situations of implants 1 in the jawbone with or without connecting element 2, wherein the surfaces, labelled by A, of the connecting elements have micromechanical anchoring positions with a biomimetically deposited apatite layer.

FIG. 7: cross section (without perspective) of a jaw area comprising gums 5a, gingiva 5a and jawbone 5b, showing the area 6 of connecting element 2 or implant 1 (prosthetic moulding, respectively) in the gums 5a of the epithelial cuff (junctional epithelium), wherein the surfaces, labelled by A, of the connecting elements have micromechanical anchoring positions with a biomimetically deposited apatite layer.

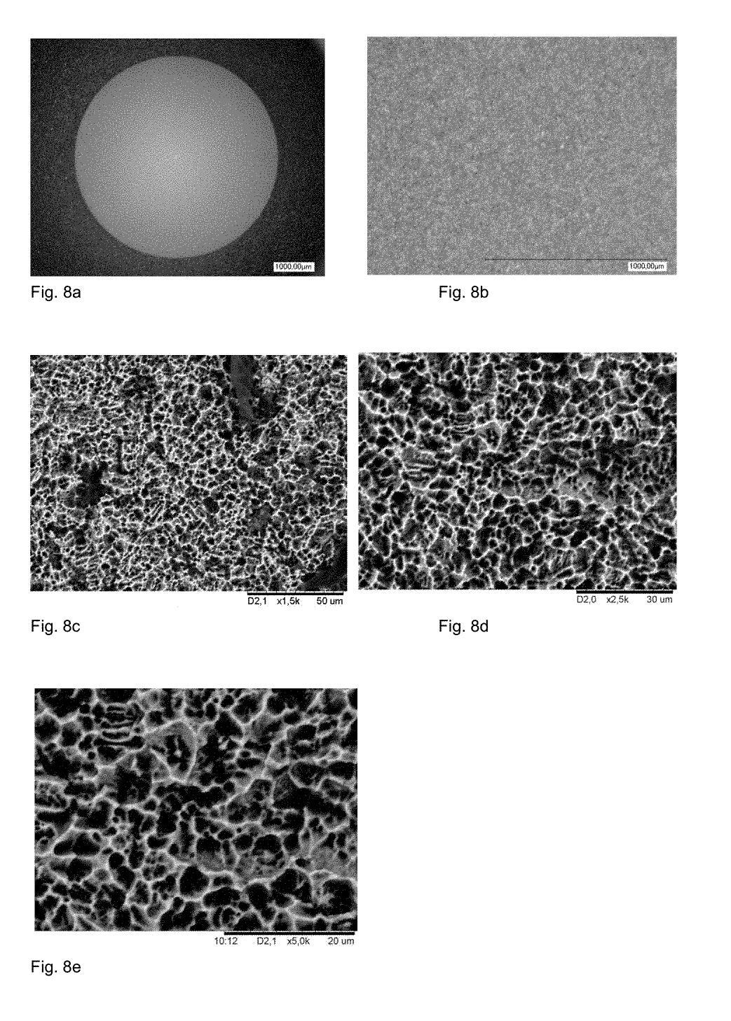

FIGS. 8a to 8e: titanium surface; FIG. 8a: non-enlarged and FIGS. 8b to 8e: enlarged (bar=1000 micrometers (FIG. 8b), =50 micrometers (μm) (FIG. 8c), 30 micrometers (FIG. 8d) and 20 micrometers (FIG. 8e)).

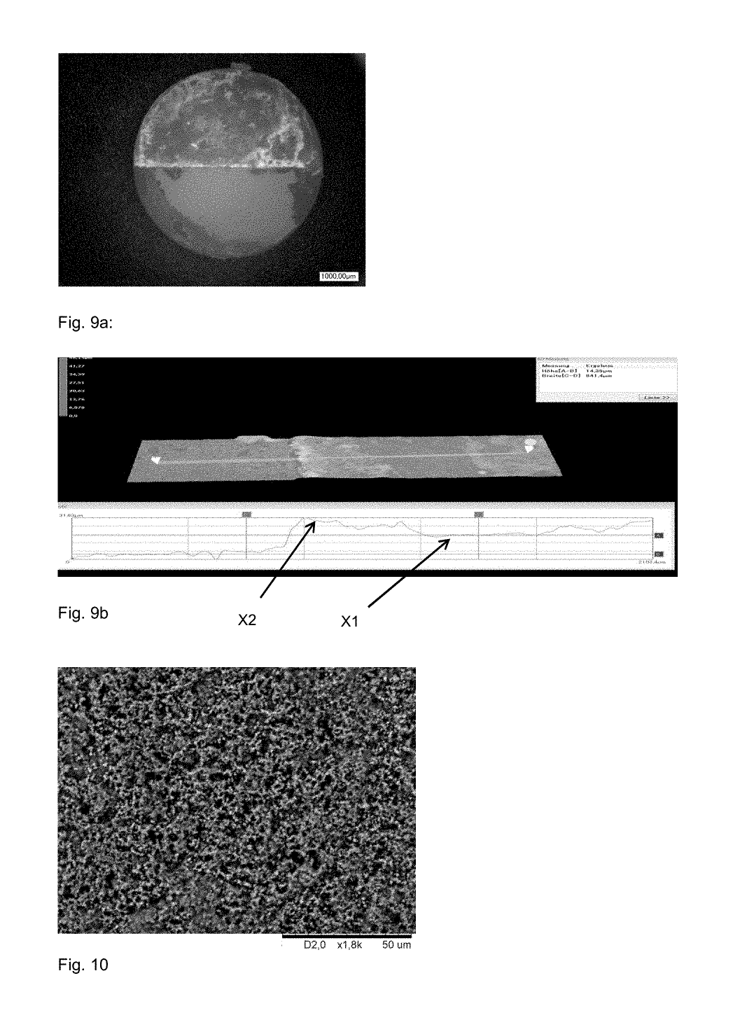

FIG. 9a: 7-fold coating on one side, layer thickness 14 to 30 μm

FIG. 9b: layer thickness of the biomimetic apatite layer: X1: ca. 14 μm, X2: ca. 30 μm

FIG. 10: REM picture of the biomimetic apatite layers (largely parallel arrangement of the almost vertical needles; bar=50 μm)

FIG. 11: enlargement of deposited biomimetic apatite layers

### EXEMPLARY EMBODIMENTS

In the following, production of the gels is described in performed experiment. Basically, cross linking with GDA (glutardialdehyde) is not required for in vitro coating, but optionally possible.

2C (two-component)—method example: (recipe for coating of Ti small plates having micromechanical anchoring positions)

### Pretreatment Composition:

For the pretreatment composition, 0.1 mol Tris buffer is added to a 1 molar calcium chloride solution setting the pH value to 9.0.

### Composition Containing Ca-Ions:

(i) For the Ca-gel: solving of 147 g CaCl₂×2H₂O with 47.5 g lactic acid in 800 ml water. A pH value of 10.5 is set using 106 ml 5 N NaOH. For the production of the gel, 18 ml of said solution are mixed with 6 g glycerin and 8 g calcium sulfate and 13.6 g 300 Bloom gelatine and heated. The liquid gel is spread with a squeegee to a thickness of 1 mm or pressed in a template having a wall thickness of 1 mm. Subsequent to solidifying the strips are cut into squares of 1×1 cm.

(ii) 2 g of a 25% GDA solution (glutardialdehyde) is topped up with water to 100 ml and gel squares are bathed therein for 20 s. Subsequently, the adhered liquid is carefully blown away. Now, the gels are analogously treated from the other side. The gel squares are shrink-wrapped in aluminium bags and individually harvested right before application.

### Composition Containing Phosphate Ions:

(i) 59 g Na₂HPO₄ is set to a pH value of 4.0 with 91 g lactic acid, 6.6 g Olaflur, 6 ml 5 N NaOH, 300 ml water and topped up to 500 ml. 24 ml of the solution and 6 g glycerin and 10 g of a 300 Bloom pork rind gelatine are prepared into a viscous solution by heating. A little liquid is inserted in a template having a wall thickness of 500 μm and pressed under 2 bar pressure. Subsequent to solidifying, the strips are removed from the template and cut into squares of 1×1 cm.

(ii) 1.5 g of a 25% GDA solution is topped up with water to 100 ml and gel squares are bathed therein for 20 s. Subsequently, the adhered liquid is carefully blown away. Now, the gels are analogously treated from the other side. The gel squares are shrink-wrapped in aluminium bags and individually harvested right before application.

### Coating of Ti Small Plates:

The surface of the Ti small plates was previously treated in non-thermic plasma (cold plasma). FIGS. 8a to 8e show the titanium surface, FIG. 8a non-enlarged and FIGS. 8b to 8e show the strongly porous surface being enlarged (bar=1000 μm (FIG. 8b), =50 μm (FIG. 8c), =30 μm (FIG. 8d) and =20 μm (FIG. 8e)). The surface exhibits a comprehensively distributed, micromechanical anchoring positions in the form of a porous surface. The pore diameters are in a range of about 0.5 μm to 20 μm.

6 titanium small plates, respectively, are treated with the pretreatment composition and coated with each a piece of phosphate gel and a piece of calcium gel for evaluation of mineralisation activity. In order to emphasize the morphological modification of the titanium surface, one half of the slice was masked so that it may only remineralise on one side. The samples are stored in climatic chamber at 37° C. and 95% humidity and were cleaned with lukewarm water and soft toothbrush after 8 to 12 hours.

Pictures of the surface were taken after 7, 10, 12 and 17 replacement intervals by means of 3D microscope (Keyence) and the layer thickness were determined from the difference in height between coated and uncoated side. Subsequently, the sample was brightly polished carefully using 4000 sand paper and the layer thickness was determined again. Completeness of coating was analysed by means of REM.

| Number of replacement | | |
|---|---|---|
| intervals | Layer thickness biomimetic apatite [μm] | |
| 1 | Almost continuous coating (non-opening type) | |
| 7 | 14 to 31 | |
| 10 | 18 to 42 | |
| 12 | 18 to 50 | |
| 17 | 20 to 60 | |
| 21* | 24 to 30 | mean 28 [μm] |

*polishing ensues after 17 replacement intervals subsequently 4 further replacement intervals

The still uncoated titanium surface having micromechanical anchoring positions in the form of a strongly porous surface structure comprising pore diameters of 1 to 7 μm, see FIG. 8e, is shown in various enlargements in FIGS. 8a to 8e. FIGS. 9a and 9b show titanium surfaces coated on one side with biomimetic apatite after 7 replacement intervals. The layer thickness of the apatite layer is at 14 to 30 μm. FIG. 10 shows an REM picture of the biomimetically deposited apatite layers according to the invention and largely parallel arrangement of the almost vertical needles. Arrangement of the crystallite needles to apatite layers of the biomimetic apatite is clearly shown in the REM picture of FIG. 11.

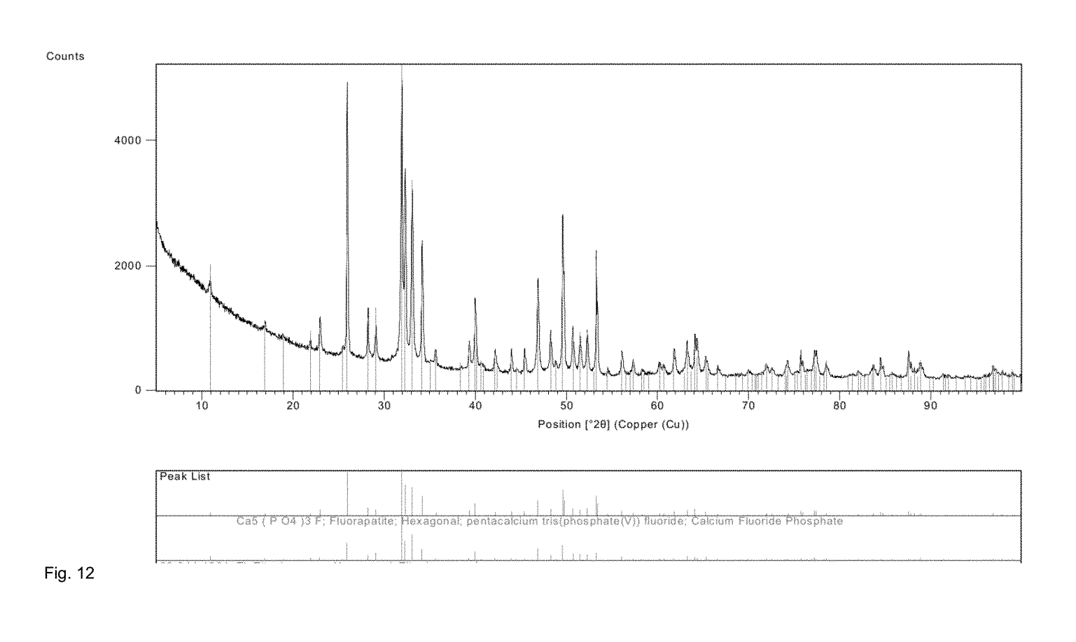

XRD diffractograms (5° to 100° (2Theta)) were acquired in reflection with X'Pert Pro MPD from biomimetically deposited fluorine apatite layers, which may unambiguously be assigned to crystalline fluorapatite. FIG. 12 shows a XRD diffractrogram of fluorapatite crystallised from gelatine as fluorapatite with gelatine (source of radiation: Copper (Cu)). The XRD lines may unambiguously be assigned to Ca₅(PO₄)₃F, fluorapatite (hexagonal). Subsequently, the samples were analysed in elementary analyzer LECO “RC-612” regarding gelatine being present due to biomimetically deposited fluorapatite. The carbon content in biomimetically deposited apatite could be determined at 0.5% by weight.

### LIST OF REFERENCE NUMERALS

0 edentulous area in the jaw
1 implant
2 connecting element (abutment, spacer, pillar, post, implant shoulder etc.) optionally with connecting screw
3 mounting element
4 upper, outer prosthetic structure, for example dental crown, superstructure optionally with outer coating
5 jaw area comprising gums and jawbone;
5a gums, gingiva; 5b: bone
6 area of the implant and/or connecting element in the gums
7 fixation screw
8 prosthetic restoration comprising 1, 2 optionally 3, as well as 4, 6 and 7
A surface having micromechanical anchoring positions, such as, for example, a porous area, a roughed, etched or an area mechanically treated with solid particles, and having at least one layer of biomimetically deposited apatite

The prosthetic mouldings according to the invention comprising an apatite layer, which preferably is arranged in the patient in the epithelial cuff (junctional epithelium) serves for lastingly and biologically durably linking an edentulous area 0, such as shown in FIG. 1a, with an implant 1 supported crown 4 (FIGS. 1b and 1c). Said linkage is dynamic. It may therefore be broken and may coalesce again. Using prosthetic mouldings according to the inventions enables attachment of the mouldings to gingiva epithelial cells in the epithelial cuff (junctional epithelium) by means of hemidesmosomes. Bacterial contamination in the area of the implant may be reduced by this measure and biological linkage to this point of contact may be obtained. FIG. 2 shows a situation without apatite layer and with apatite layer according to the invention in FIG. 7. The prosthetic moulding according to the invention, here the connecting element 2, has a biomimetically deposited apatite layer according to the invention in the area of the gums 5a of the epithelial cuff (junctional epithelium) 6. Said biomimetically deposited apatite layer preferably comprises a content of amino acids, amino acid derivatives, proteins, denatured collagen, in particular the apatite comprises components of collagen, denatured collagen, gelatine protein chains, gelatine glycerin gel of the compositions from which the apatite is deposited.

FIGS. 4 and 6 show different installation situations of implants 1 with different connecting elements 2, wherein in FIG. 6, the connecting elements 2 have different areas, labelled by A, of the surface. Said areas A have micromechanical anchoring positions, such as, for example, a porous area, a roughened, etched or an area mechanically treated with solid particles. A biomimetic apatite layer of at least 1 μm (micrometer) to 100 μm or to 1 mm is grown up on these micromechanical anchoring positions. According to the invention, the biomimetic apatite layer is obtained by crystallisation of apatite from the afore-mentioned compositions, in particular gels. The biomimetic apatite is later, in the mouth of the patient, linked to the epithelial cells of the gingiva, in particular the epithelial cuff (junctional epithelium), by cellular attachment, in particular via hemidesmosomes.

FIGS. 3 and 5 show typical mouldings of a prosthetic tooth restauration, wherein FIG. 3 shows the particular mouldings and FIG. 5 the assembled prosthetic mouldings for insertion in an upper jaw.

The person skilled in the art knows that, according to the invention, all appropriate mouldings made of every physiologically suitable material may be provided with a biomimetic apatite layer as prosthetic mouldings. Absorbable mouldings may also be used as prosthetic mouldings.

1. A prosthetic moulding, wherein the moulding has, at least area by area, at least one layer of biomimetic apatite selected from fluorapatite, hydroxylapatite or their mixtures on its surface, wherein the surface has micromechanical anchoring positions at least in this area.

2. The moulding according to claim 1, wherein the layer thickness of the at least one apatite layer is at least 1 μm.

3. The moulding according to claim 1, wherein the moulding is an enossal implant (1), dental, enossal (intraosseous) implant (1), connecting element (2), mounting element (3), dental sleeve, abutment (2), suprastructure (2), part of a dental prosthesis, total denture, orthopedic prosthesis or parts thereof, artificial tooth, veneer, inlay, onlay, dental supporting structure (2,3), bridge (4), crown (4), relining, denture saddle, bone prosthesis, joint prosthesis, revision total joint endoprosthesis, and/or spacer.

4. The moulding according to claim 1, wherein the moulding is a connecting element (2), a mounting element (3), dental, enossal dental implant (1), and/or an implant post (2).

5. The moulding according to claim 1, wherein the moulding is formed of titanium, titanium alloy, titanium oxide, cobalt-chromium alloy, CoCrMo alloy, gold, dental ceramic, zirconium oxide, lithium disilicate, polymer, polymer mixture, dental prosthetic plastic.

6. The moulding according to claim 1, wherein the biomimetic apatite comprises amino acids, amino acid derivatives, proteins and/or denatured collagen.

7. The moulding according to claim 1, wherein at least one area of the surface of the moulding has micromechanical anchoring positions, which comprise a porous and/or rough surface topography.

8. The moulding, according to claim 1, wherein
a) the surface of the prosthetic moulding has, area by area, micromechanical anchoring positions in the area, which is later arranged in the area of the gums (6),
b) the surface of the prosthetic moulding has, area by area, micromechanical anchoring positions in the area, which is later arranged in the area of epithelial cells, of the gums, of the gingiva, gingival epithelial cells, fibroblasts, or the area of the epithelial cuff (junctional epithelium), wherein the prosthetic moulding is selected from connecting element (2), mounting element (3), abutment, implant (1), upper, outer prosthetic structure (4), crown (4), suprastructure (4), enossal implant (1), dental enossal (intraosseous) implant (1), dental sleeve, part of a dental prosthesis, part of a bone prosthesis, total denture, orthopedic prosthesis or parts thereof, artificial tooth, veneer, inlay, onlay, dental supporting structure (2,3), bridge (4), relining, denture saddle, bone prosthesis, joint prosthesis, revision total joint endoprosthesis, and/or spacer.

9. The moulding according to claim 1, wherein the biomimetic apatite is selected from fluorapatite, hydroxylapatite or their mixtures and has at least a carbon content of in the range of 0.25 to 2.5% by weight.

10. A method for the deposition of biomimetic apatite selected from fluorapatite, hydroxylapatite or their mixtures on a prosthetic moulding, comprising the steps of
(i.a) providing a prosthetic moulding, wherein at least one area of the surface of the moulding has micromechanical anchoring positions, or
(i.b) treating at least one area of the surface of the prosthetic moulding, and obtaining micromechanical anchoring positions, and optional
(ii) treating at least one area of the surface of a prosthetic moulding, with a pretreatment compositions having a defined pH value,
(iii) contacting at least this area of the prosthetic moulding comprising micromechanical anchoring positions with a composition containing phosphate ions, which comprises a gel forming agent, whereby a gel layer is formed,
(iv) optionally applying a further layer, contacting the first gel layer with a further composition which comprises a gel forming agent, forming a further gel layer,
(v) contacting the first gel layer or the further layer with a composition containing calcium ions, whereby a gel layer is formed,
(vi) depositing biomimetic apatite selected from fluorapatite, hydroxylapatite or their mixtures on the surface of the prosthetic moulding.

11. The method according to claim 10, wherein
(a) the composition containing phosphate ions comprises,
(a.1) at least one gel forming agent,
(a.2) water-soluble phosphates or phosphates being hydrolysable to water-soluble phosphate ions,
(a.3) optionally fluoride,
(a.4) optionally a carboxylic acid or a buffer system of pH 4 to 7,
(a.5) optionally glycerin, and
(b) wherein the composition containing calcium ions comprises,
(b.1) at least one gel forming agent
(b.2) calcium ions,
(b.3) optionally glycerin.

12. The method according to claim 10, wherein treating the surface of the moulding comprises mechanical, chemical, electrochemical treatment and/or treating in a plasma process or by a combination of the methods, and an area of the surface of the prosthetic moulding having micromechanical anchoring positions is obtained, and optionally the surface is activated by a chemical, electrochemical and/or in a plasma process.

13. The method according to claim 10, wherein the composition containing phosphate ions and/or calcium ions, each independently, comprises amino acids, derivatives of amino acids, proteins collagen, or denatured collagen.

14. The method according to claim 10, wherein the gel forming agent comprises gelatine.

15. The method according to claim 10, wherein the composition containing phosphate ions and/or calcium ions, each independently, has a content of water.

16. An intermediate, comprising a prosthetic moulding, wherein
the moulding has, at least area by area, at least one gel layer of a composition containing phosphate ions on its surface, and optionally thereon a further gel layer, and optionally a gel layer of a composition containing calcium ions.

17. A prosthetic moulding obtainable by a method according to claim 10.

18. Method of using biomimetic apatite for coating, at least area by area, of prosthetic mouldings.

19. Method of using a composition containing phosphate ions and of a composition containing calcium ions according to claim 10, or of formulations containing these compositions, for biomimetic deposition of apatite on a surface of a prosthetic moulding, wherein the surface has micromechanical anchoring positions or, wherein the surface of the moulding has been activated mechanically, chemically, electrochemically and/or by means of a plasma process prior to deposition.

20. Method according to claim 19, characterised in that a deposition, at least area by area, of biomimetic apatite ensues, and/or an essentially homogenous depositions of apatite ensues in this area.

21. A moulding according to claim 1 for use in dental, prosthetic, or surgical treatment for tooth loss for cellular attachment of cells of the gums or mucosal cells of the gums via hemidesmosomes or other biological mechanisms to areas contacting the moulding.
